(19) United States
(12) Patent Application Publication
(10) Pub. No.: US 2015/0080832 A1
(43) Pub. Date: Mar. 19, 2015

(54) Overnight Diaper Insert

(71) Applicants: Harold Edward Thomas, Gallatin, TN (US); Crystal Bridgers Thomas, Charlotte, NC (US)

(72) Inventors: Harold Edward Thomas, Gallatin, TN (US); Crystal Bridgers Thomas, Charlotte, NC (US)

(21) Appl. No.: 14/536,569

(22) Filed: Nov. 7, 2014

Related U.S. Application Data
(60) Provisional application No. 61/962,681, filed on Nov. 14, 2013.

(51) Int. Cl. A61F 13/47; A61F 13/62; A61F 13/515; A61F 13/56

(52) U.S. Cl. CPC A61F 13/47; A61F 13/5611; A61F 13/625; A61F 13/515
USPC 604/385.03; 604/385.01

(57) ABSTRACT

An absorbent pad useful as a diaper insert to augment the absorbent capacity of a disposable or non-disposable diaper, which comprises two superposed fluid pervious layers and a liquid absorbent layer disposed between the liquid pervious layers. The absorbent pad first receives liquid excrement from the wearer and stores this in the absorbent layer. As the absorbent capacity of the absorbent pad is reached, liquid migrates from the absorbent pad into the absorbent region of the primary diaper thus increasing the total capacity of the system.

### CROSS-REFERENCE TO RELATED APPLICATIONS

This application claims the benefit of the filing date of provisional application 61/962,681.

### BACKGROUND OF THE INVENTION

The present invention relates to diapers and more particularly to disposable inserts for disposable and non-disposable diapers. The diaper inserts of the present invention increase the total absorbent capacity of the diaper resulting in extended wearing time and reduced leakage especially in overnight situations.

Disposable diapers have been known in the art for many years, and more recently disposable diapers with increased absorbent capacity have been available specifically for overnight use. However, these diapers do not always provide the necessary capacity for many children and can still result in some leakage since they are designed to accommodate general physical size of the child and not necessarily the full range of fluid handling requirements. Furthermore, cloth diaper absorbency is generally limited to the amount of cloth used, and increasing the amount or “double diapering” can decrease the comfort of the wearer.

In the past various diaper inserts have been disclosed with the intent of being used as a removable absorbent pad to prolong the useful life of a disposable diaper. These products however, do not effectively add to the total absorbent capacity of the combined system since they are designed to be self containing and to be removed while the primary diaper remains in place. This in itself defeats the purpose of extending the absorbent capacity of the system to allow for longer, uninterrupted sleep.

It has also been noted in various social media forums, that some parents will resort to inserting an adult incontinent pad in a disposable or cloth diaper in an attempt to ameliorate the problems of leakage and premature awakening. However, since these products are intended to contain all liquid they are subjected to without leakage and are comprised of a fluid impermeable layer on the outside to prevent such leakage into clothing, they cannot allow liquid transmission to the primary diaper and thus prevent full utilization of the combined absorbent structures.

### SUMMARY OF THE INVENTION

It is therefore an object of the present invention to provide an insert that can augment the absorbent capacity of the primary diaper by first receiving liquid excrement and then allowing the liquid to migrate through the insert and eventually into the primary diaper thus enabling complete use of the combined absorbent capacities and minimizing or eliminating leakage over extended time frames.

Another object is to provide a range of sizes and capacities of inserts so that the parent or caregiver can customize the total level of absorbent capacity required without having to utilize different primary diapers for overnight protection.

Another object is to allow for secured placement of the diaper insert by means of an adhesive applied on the face contacting the primary diaper topsheet. This prevents the insert from moving out of place during diaper placement and movement of the child.

Another object is to allow for secured placement of the diaper insert by means of male component of a “hook and loop” fastener that attaches to the diaper topsheet. This prevents the insert from moving out of place during diaper placement and movement of the child.

### BRIEF DESCRIPTION OF THE DRAWINGS

These and other objects and advantages of the invention will become apparent upon consideration of the following description and drawings.

FIG. 1 is a plan view of the overnight insert made in accordance with the present invention.

FIG. 2 is a cross section of the overnight insert along line of A-A of FIG. 1.

FIG. 3 is a cross section along the line of B-B of FIG. 4 of the overnight insert positioned on the diaper topsheet or bodyside lining.

FIG. 4 is a plan view of the overnight insert positioned on the diaper topsheet between the barrier leg cuffs.

### DETAILED DESCRIPTION OF THE INVENTION

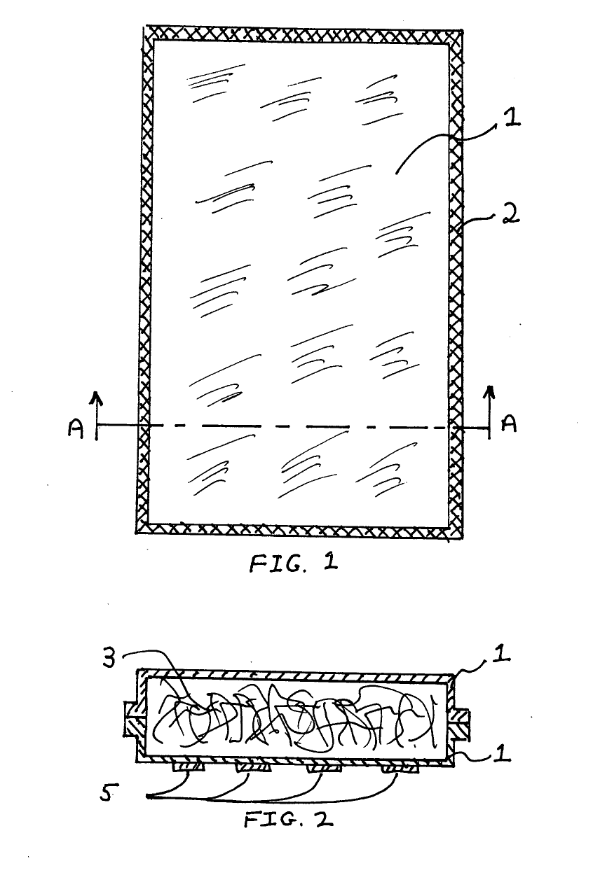

The preferred embodiment of the present invention is shown in FIG. 2 wherein the overnight insert is comprised of an absorbent layer or pad 3 covered on both sides by a thin, liquid permeable fabric 1 such as a nonwoven sheet. Such nonwoven sheets are typically made from polypropylene and treated with a surfactant to improve their hydrophilicity and hence fluid permeability. The edges of the absorbent layer should be positioned slightly inside the edges of the juxtaposed liquid permeable fabrics to facilitate sealing of the edges. The layers of liquid permeable fabric are bonded together 2 around the periphery of the insert as shown as in FIG. 1 using any one of several methods including adhesives, thermal welding and ultrasonic bonding. Furthermore, although the insert of the present invention is shown as rectangular in shape, any anatomically designed configuration is possible including those that may be designed specifically for male or female children. Contrary to conventional inserts and incontinent pads which comprise at least one hydrophobic, liquid impermeable layer, the current invention consists of liquid permeable layers 1 on both surfaces to facilitate liquid migration over time through the insert, through the liquid permeable diaper topsheet 7 and into the absorbent core 8 of the diaper while retaining the absorbent core components 3 within the confines of the insert.

The absorbent pad 3 in FIG. 2 can be constructed from a variety of materials including widely used cellulosic fibers or wood pulp as well as organic hydrophilic fibers and organic non-cellulosic particles. The absorbent pad can be designed with different weights, thickness, loft and densities to optimize the fluid acquisition, retention and transport as necessary.

In use, the overnight insert of the present invention is positioned on top of the diaper body liner or topsheet 7 and between the diaper barrier leg cuffs 6. In the preferred embodiment, the insert is held in position by one of several means 5 to ensure the insert remains in place and movement during sleep is minimized. FIG. 2 depicts such attachment features as 5. These can be peel and stick adhesive systems or male components of a hook and loop system that will engage the diaper topsheet. These same fixation means are also applicable when the overnight insert is used in conjunction with a conventional cloth diaper. For completeness, the liquid impermeable diaper backsheet 9, the frontal closure tabs 10 and the rear closure tabs 11 are also shown.

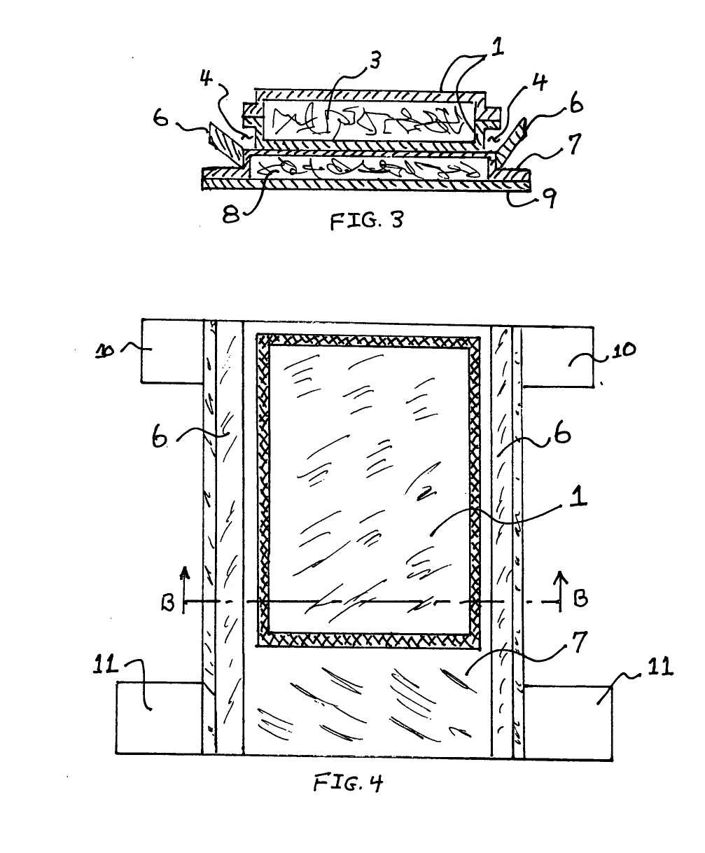

Using the following procedure, the absorbent capacity of several conventional disposable diapers and incontinent pads was determined. A 0.9% saline solution was used to simulate actual urine. This was poured into a pan and the subject diaper or pad was placed in the solution and allowed to absorb the liquid for 10 minutes. Then the diaper or pad was removed and held vertically for 2 minutes allowing excess liquid to drain. The diaper or pad was weighted dry, fully saturated and after draining to determine the absorbent capacity. The products that were evaluated were: Parent's Choice™ Diapers Size 5 with “ultra absorbent core” available from Walmart, Pampers® Baby Dry 12 hour size 5 made by Procter & Gamble, Huggies® Overnight Size 5 made by Kimberly Clark, Tena Serenity® Heavy-Long incontinent pads made by SCA, and Prevail® Moderate Long incontinent pads made by First Quality. When weighing the incontinent pads, the release paper on the film side was removed. Results of these measurements are shown in Table 1. Using this data for comparisons, several examples of the present invention were then constructed and evaluated. Absorbent capacity of diapers containing examples of the present invention was determined by the following procedure. The overnight insert was placed on the diaper topsheet 7 between the barrier leg cuffs 6 with a predisposition towards the front edge of the diaper as it would be worn by the infant. The diaper with insert installed was then placed in a pan. Saline solution as described previously was then poured gently onto the insert topsheet being careful to not allow any runoff past the edges of the insert. This was done for 10 minutes or until liquid could be observed standing between the insert edge and the diaper barrier leg cuff at location 4 in FIG. 3. The combined insert and diaper were then held vertically for 2 minutes and allowed to drain. The diaper and insert were weighed dry, fully saturated and after draining to determine the absorbent capacity of the system.

TABLE 1

| | Dry Wt | Wet Wt: | Absorbent | Abs/ |
|---|---|---|---|---|
| | (g): | (g) | Capacity (g): | Dry |
| Parents Choice Diaper Size 5 | 27.3 | 461.7 | 434.3 | 15.9 |
| Pampers Baby Dry Size 5 | 33.3 | 521.0 | 487.7 | 14.6 |
| Huggies Size 5 Overnight | 47.7 | 691.3 | 643.7 | 13.5 |
| Tena Senenity Heavy-Long | 30.7 | 535.0 | 504.3 | 16.4 |
| Prevail Moderate long | 24.3 | 459.0 | 434.7 | 17.9 |

### Example 1

An example of the present invention was achieved by removing the liquid impermeable film backsheet from a Tena incontinent pad and placing the exposed absorbent pad directly on the topsheet or body side liner of a Parent's Choice size 5 diaper. The diaper and pad were then placed in a pan and the 0.9% saline solution was poured gradually onto the Tena topsheet. Although this test protocol called for a 10 minute period for introducing the liquid to the absorbent system, the now uncontained absorbent core of the Tena pad began to fall apart and leak.

### Example 2

Another example of the present invention was constructed by cutting 4 rows of 1″ long slits in the liquid impermeable film backsheet of a Tena incontinent pad between the adhesive stripes after removing the release paper. This pad was then placed on the topsheet of a Parents Choice Size 5 diaper and between the diaper barrier leg cuffs as indicated in FIG. 4. During fabrication it was noted that super absorbent particles within the absorbent core could escape through the slits in the film.

### Example 3

In yet another and more preferred example of the present invention, the topsheet was removed from a Tena incontinent pad. The film backsheet was removed from another Tena pad and replaced with the aforementioned topsheet by gluing the new topsheet to the pad along its periphery using a hot melt adhesive. This example was constructed with a liquid permeable nonwoven fabric on both sides of the absorbent pad and eliminated the issue of particulate loss through the slits in the film of example 2.

### Example 4

In a further example of the preferred construction, the liquid impermeable film backsheet of a Prevail incontinent pad was removed and replaced with the nonwoven topsheet removed from another Prevail pad with the method as described in Example 3.

### Example 5

As seen in Table 2, Example 4 exhibited a lower than expected % of theoretical absorbent capacity. This may have been that due to the smaller insert, there was insufficient time for the fluid to fully be absorbed by the diaper's absorbent core. Example 4 was repeated, but the duration of time for absorbing the fluid was extended from 10 minutes to 25 minutes. As can be seen in Table 2, this system then also approaches theoretical maximum capacity.

All examples were evaluated for total absorbent capacity and the results are shown in Table 2.

TABLE 2

| | | | Absorbent | | | |
|---|---|---|---|---|---|---|
| | Dry Wt | Wet Wt | Capacity | | Theor | % of |
| | (g) | (g) | (g) | Abs/Dry | Cap | Theor |
| Example 1 | 61 | 661 | 600 | 9.8 | 1019 | 59% |
| Example 2 | 61 | 976 | 932 | 15.3 | 939 | 99% |
| Example 3 | 62 | 987 | 915 | 14.7 | 939 | 98% |
| Example 4 | 52 | 793 | 743 | 14.3 | 869 | 85% |
| Example 5 | 54 | 898 | 843 | 15.7 | 869 | 97% |

The examples set forth above are not intended to represent the full breadth of the present invention, but serve to demonstrate its practicality and usefulness. The description of the preferred embodiment of the present invention is intended to be illustrative and not restrictive. Details concerning selection of specific materials, their dimensions, fabrication and enhancements should be clear to one skilled in the art and these may be modified or changed without departing from the scope or spirit of the invention.

What is claimed is:

1. A disposable absorbent pad comprising:
first and second superposed fluid pervious layers having a length and a width and a liquid absorbent layer disposed between said fluid pervious layers wherein the length and width of said liquid absorbent layer are less than that of said first and second fluid pervious layers and said liquid absorbent layer is positioned inside the edges of said fluid pervious layers and the edges of said fluid pervious layers are secured to each other thus containing said liquid absorbent layer between said first and second fluid pervious layers and providing a seal around the pad perimeter, said pad being adapted to be inserted in a disposable or cloth diaper such that the first said fluid pervious layer is in contact with the body side liner of the diaper and the second said fluid pervious layer is in contact with the wearer.

2. A disposable absorbent pad as defined in claim 1 wherein the liquid absorbent layer contains organic non-cellulosic particles.

3. A disposable absorbent pad as defined in claim 1 wherein the first and second fluid pervious layers comprise nonwoven sheets.

4. A disposable absorbent pad comprising:
first and second superposed fluid pervious layers having a length and a width and a liquid absorbent layer disposed between said fluid pervious layers wherein the length and width of said liquid absorbent layer are less than that of said first and second fluid pervious layers and said liquid absorbent layer is positioned inside the edges of said fluid pervious layers and the edges of said fluid pervious layers are secured to each other thus containing said liquid absorbent layer between said first and second fluid pervious layers and providing a seal around the pad perimeter, said pad being adapted to be inserted in a disposable or cloth diaper such that the first said fluid pervious layer is in contact with the body side liner of the diaper and the second said fluid pervious layer is in contact with the wearer; and
an adhesive is applied to the first said fluid pervious layer to allow adherence to the body side layer of the diaper.

5. A disposable absorbent pad as defined in claim 4 wherein the liquid absorbent layer contains organic non-cellulosic particles.

6. A disposable absorbent pad as defined in claim 4 wherein the first and second fluid pervious layers comprise nonwoven sheets.

7. A disposable absorbent pad comprising:
first and second superposed fluid pervious layers having a length and a width and a liquid absorbent layer disposed between said fluid pervious layers wherein the length and width of said liquid absorbent layer are less than that of said first and second fluid pervious layers and said liquid absorbent layer is positioned inside the edges of said fluid pervious layers and the edges of said fluid pervious layers are secured to each other thus containing said liquid absorbent layer between said first and second fluid pervious layers and providing a seal around the pad perimeter, said pad being adapted to be inserted in a disposable or cloth diaper such that the first said fluid pervious layer is in contact with the body side liner of the diaper and the second said fluid pervious layer is in contact with the wearer; and a male hook component is applied to the first said fluid pervious layer to allow adherence to the body side layer of the diaper.

8. A disposable absorbent pad as defined in claim 7 wherein the liquid absorbent layer contains organic non-cellulosic particles.

9. A disposable absorbent pad as defined in claim 7 wherein the first and second fluid pervious layers comprise nonwoven sheets.
